(19) United States
(12) Patent Application Publication
(10) Pub. No.: US 2008/0020478 A1
(43) Pub. Date: Jan. 24, 2008

(54) Method of Detecting an Analyte Using a Holographic Sensor

(76) Inventors: Christopher Robin Lowe, Cambridge (GB); Satyamoorthy Kabilan, Cambridge (GB); Mei-Ching Lee, London (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/597,983

(22) PCT Filed: Jun. 6, 2005
(86) PCT No.: PCT/GB05/02222

§ 371 Date: Aug. 10, 2007

(30) Foreign Application Priority Data
Jun. 7, 2004 (GB) 0412654.6

(51) Int. Cl. G01N 33/66; A61B 3/10; G01N 21/00; G02B 5/32; G01N 21/01

(52) U.S. Cl. 436/127; 351/219; 359/2; 422/82.05; 436/164

(57) ABSTRACT

A method for the detection of an analyte in a fluid, which comprises contacting the fluid with a holographic element comprising a medium and a hologram disposed throughout the volume of the medium, wherein an optical characteristic of the element changes as a result of a variation of a physical property occurring throughout the volume of the medium, and wherein the variation arises as a result of interaction between the medium and the analyte; and detecting any change of the optical characteristic of the element; wherein (a) the medium comprises a group which is capable of reacting with the analyte, wherein the analyte or the group is capable of existing in a plurality of forms, and the detecting is conducted in the presence of a first catalyst which is capable of catalysing the conversion of a relatively less reactive form of the analyte or group to a relatively more reactive form; or (b) the fluid comprises a component, other than the analyte, which is capable of interacting with the medium, and the detecting is conducted in the presence of a second catalyst capable of catalysing the removal of said component.

### FIELD OF THE INVENTION

This invention relates to a method for the detection of an analyte using a holographic sensor.

### BACKGROUND TO THE INVENTION

WO9526499 discloses a holographic sensor for the detection of an analyte. This sensor comprises a holographic element comprising a support medium and a hologram disposed throughout the volume of the medium. An optical characteristic of the element changes a result of a variation of a physical property occurring throughout the volume of the medium, the variation arising as a result of reaction between the medium and the analyte. By monitoring any change in the optical characteristic, the presence of the analyte can be detected. WO03/087789 describes a process for the continuous sensing of an analyte using a holographic sensor.

A particular analyte of interest is glucose. There is a need for minimally invasive, easy-to-use glucose sensors, particularly ophthalmic glucose sensors. The concentration of glucose in the blood is typically of the order of 20 mM, whereas in the eye it is about 0.1 mM. The levels of glucose in the eye are known to correlate to those in the blood. Thus, blood levels of glucose can be monitored indirectly by measuring the levels in an ocular fluid such as tears.

Glucose (also known as D-glucose) occurs in five different forms. The four cyclic forms of glucose, namely α-D-glucopyranose, β-D-glucopyranose, α-D-glucofuranose and β-D-glucofuranose, coexist in equilibrium with the acyclic form, D-glucose aldehyde, via a process called “complex mutarotation”. Typically, the proportions of the α-D-glucopyranose, β-D-glucopyranose, α-D-glucofuranose, β-D-glucofuranose and D-glucose aldehyde are about 39.4, 60.2, 0.2, 0.2 and 0.001% respectively (Shoji et al, J. Am. Chem. Soc., 124(42), 12486-93).

A well-documented reaction is that of glucose with boronic acid compounds. It has been suggested that the binding of glucose to a boronic acid, RB(OH)₂, occurs only when the glucose is in the α-D-glucofuranose form (Shoji et al). Other studies (Shiomi et al, J. Chem. Soc. Perkin Trans. 1, 2111-2117), however, postulate that the α-D-pyranose form that may also bind, provided the NMR coupling constants are assigned correctly. Furthermore, it has been suggested that the boronic acid must be in a tetrahedral (i.e. RB(OH)₃⁻), as opposed to a trigonal, conformation. It has been suggested that boronic acids preferentially bind to diols which are in a cis conformation (Liu et al, J. Organomet. Chem., 493(1-2), 91-94). The reaction is fully reversible, the pH at which the conformational change occurs strongly influenced by the structure of R. R is preferably a phenyl group or derivative thereof. Generally, only a small proportion of the α-D-glucofuranose form is present, and so little reaction takes place, often at a low rate.

The extent of reaction between glucose and a boronic acid can be increased by varying the extent of complex mutarotation. The enzyme mutarotase catalyses the conversion of the β-forms (via the linear form) to α-D-glucofuranose. Alternatively, the extent of reaction can be increased by first converting glucose to fructose or ribose, using an enzyme such as glucose isomerase. Fructose and ribose react with boronic acids in an analogous manner to glucose.

### SUMMARY OF THE INVENTION

The present invention is based upon the realisation that the response of a holographic sensor can be increased by detecting any interaction between the holographic support medium and analyte in the presence of an agent, more specifically a catalyst, which enhances that interaction. For example, a holographic sensor comprising pendant boronic acid groups may be used for the detection of glucose. However, since the levels of the α-D-glucofuranose form are generally very low, the time and level of response of such a sensor may be poor. The response may be dramatically enhanced by carrying out detection in the presence an enzyme such as mutarotase or glucose isomerase.

A first aspect of the invention is a method for the detection of an analyte in a fluid, which comprises contacting the fluid with a holographic element comprising a medium and a hologram disposed throughout the volume of the medium, wherein an optical characteristic of the element changes as a result of a variation of a physical property occurring throughout the volume of the medium, and wherein the variation arises as a result of interaction between the medium and the analyte; and detecting any change of the optical characteristic of the element; wherein

(a) the medium comprises a group which is capable of reacting with the analyte, wherein the analyte or the group is capable of existing in a plurality of forms, and the detecting is conducted in the presence of a first catalyst which is capable of catalysing the conversion of a relatively less reactive form of the analyte or group to a relatively more reactive form; or

(b) the fluid comprises a component, other than the analyte, which is capable of interacting with the medium, and the detecting is conducted in the presence of a second catalyst capable of catalysing the removal of said component.

In the case of glucose, detection preferably takes place in the presence of a catalyst which catalyses the conversion of α-D-glucopyranose, β-D-glucofuranose and/or D-glucose aldehyde to α-D-glucofuranose. More preferably, detection takes place in the presence of mutarotase and/or glucose isomerase.

Another aspect of the invention is an ophthalmic device which comprises a holographic element and a catalyst as defined above. The insert may be in the form of a contact lens or implantable device.

### DESCRIPTION OF THE INVENTION

The term “glucose” as used herein refers to the known cyclic and linear forms of glucose.

The term “ophthalmic device” as used herein refers to contact lenses (both hard and soft), corneal onlays, implantable ophthalmic devices and the like.

The term “contact lens” as used herein refers to any hard or soft lens used on the eye or ocular vicinity for vision correction, diagnosis, sample collection, drug delivery, wound healing, cosmetic appearance or other ophthalmic application. The lens may be a daily-disposable, daily-wear or extended-wear lens.

The term “implantable ophthalmic device” as used herein refers to an ophthalmic device which is used in, on or about the eye or ocular vicinity. Such devices include intraocular lenses, subconjunctival lenses, intracorneal lenses, and shunts/implants (e.g. a stent or glaucoma shunt) that can rest in the cul de sac of an eye.

The interaction between the medium and the analyte may be physical and/or chemical. The sensor may allow for the continuous detection of an analyte.

The analyte may be able to exist in a plurality of forms. In this case, a catalyst may be used that catalyses the conversion of the analyte to a more reactive form. An example of such an analyte is glucose, which. via mutarotation is able to exist in five different forms. Thus, in the case of glucose, the catalyst may be an enzyme such as mutarotase or glucose isomerase, allowing the rate of conversion to α-D-glucofuranose to increase. When a medium comprising phenylboronic acid or like groups is used, the extent of reaction between glucose and the medium will be enhanced.

Lactate (lactic acid) is known to interfere with the sensing of glucose. This is a particular problem in the eye, where lactate is present at relatively high concentration. The catalyst thus may promote the removal of lactate. For example, lactate oxidase may be used. This enzyme catalyses the breakdown of lactate to (via a pyruvate intermediate) hydrogen peroxide. Hydrogen peroxide may react with silver and thus, if the sensor is silver-based, it is preferred that an enzyme such as catalase is present to remove any unwanted hydrogen peroxide produced. An alternative to lactate oxidase is lactate dehydrogenase, which converts lactic acid into pyruvate without the production of hydrogen peroxide.

Conversely, should lactate be the analyte of interest then it may be desirable to remove glucose from the system. In this case, an enzyme such as glucose oxidase may be used.

The interaction between the medium and analyte can be detected remotely, using non-ionising radiation. The extent of interaction is reflected in the degree of change of the physical property, which is detected as a variation in an optical characteristic, preferably a shift in wavelength of non-ionising radiation.

The property of the holographic element which varies may be its charge density, volume, shape, density, viscosity, strength, hardness, charge, hydrophobicity, swellability, integrity, cross-link density or any other physical property. Variation of the or each physical property, in turn, causes a variation of an optical characteristic, such as polarisability, reflectance, refractance or absorbance of the holographic element.

The hologram may be disposed on or in, part of or throughout the bulk of the volume of the support medium. An illuminating source of non-ionising radiation, for example visible light, may be used to observe variation(s) in the, or each, optical characteristic of the holographic element.

The holographic effect may be exhibited by illumination (e.g. under white light, UV or infra-red radiation), specific temperature, magnetic or pressure conditions, or particular chemical, biochemical or biological stimuli. The hologram may be an image of an object or a 2- or 3-dimensional effect, and may be in the form of a pattern which is only visible under magnification.

The hologram can be generated by the diffraction of light. The holographic element may further comprise means for producing an interference effect when illuminated with laser light and such means can comprises a depolarising layer.

More than one hologram may be supported on, or in, a holographic element. Means may be provided to detect the or each variation in radiation emanating from the or each hologram, arising as a result of a variation in the or each optical characteristic. The holographic elements may be dimensioned and arranged so as to sense two or more independent events/species and to affect, simultaneously, or otherwise, radiation in two or more different ways. Holographic elements may be provided in the form of an array.

The holographic support medium may be obtained by the polymerisation of monomers, such as (meth)acrylamide and/or (meth)acrylate-derived comonomers. In particular, the monomer HEMA (hydroxyethyl methacrylate) is readily polymerisable and cross-linkable. PolyHEMA is a versatile support material since it is swellable, hydrophilic and widely biocompatible.

Other examples of holographic support media which may be modified to include boronic acid groups are gelatin, K-carageenan, agar, agarose, polyvinyl alcohol (PVA), sol-gels (as broadly classified), hydro-gels (as broadly classified), and acrylates.

A parameter determining the response of a holographic element is the extent of cross-linking. The number of cross-linking points due to polymerisation of monomers should not be so great that complex formation between polymer and analyte-binding groups is relatively low, since the polymer film may become too rigid. This may inhibit the swelling of the support medium.

In a preferred embodiment, an insert of the invention is in the form of a contact lens. The lens may be manufactured using any suitable material known in the art. The lens material may be formed by the polymerisation of one or more monomers and optionally one or more prepolymers. The material may comprise a photoinitiator, visibility tinting agent, UV-blocking agent and/or a photosensitiser.

A preferred group of lens materials is prepolymers which are water-soluble and/or meltable. It is preferred that the material comprises one or more prepolymers which are in a substantially pure form (e.g. purified by ultrafiltration). Preferred prepolymers include water-soluble crosslinkable poly(vinyl alcohol) prepolymers (as described in U.S. Pat. No. 5,583,163 and U.S. Pat. No. 6,303,687); a water-soluble vinyl group-terminated polyurethane, obtainable by reacting an isocyanate-capped polyurethane with an ethylenically unsaturated amine (primary or secondary amine) or an ethylenically unsaturated monohydroxy compound, wherein the isocyanate-capped polyurethane can be a copolymerisation product of at least one polyalkylene glycol, a compound containing at least 2 hydroxyl groups, and at least one compound with two or more isocyanate groups; derivatives of a polyvinyl alcohol, polyethyleneimine or polyvinylamine (see, for example, U.S. Pat. No. 5,849,841); a water-soluble cross-linkable polyurea prepolymer as described in U.S. Pat. No. 6,479,587; cross-linkable polyacrylamide; cross-linkable statistical copolymers of vinyl lactam, MMA and a comonomer, as disclosed in EP0655470 and U.S. Pat. No. 5,712,356; cross-linkable copolymers of vinyl lactam, vinyl acetate and vinyl alcohol, as disclosed in EP0712867 and U.S. Pat. No. 5,665,840; polyether-polyester copolymers with cross-linkable side chains, as disclosed in EP0932635; branched polyalkylene glycol-urethane prepolymers, as disclosed in EP0958315 and U.S. Pat. No. 6,165,408; polyalkylene glycol-tetra(meth)acrylate prepolymers, as disclosed in EP0961941 and U.S. Pat. No. 6,221,303; and cross-linkable polyallylamine gluconolactone prepolymers, as disclosed in WO00/31150.

The lens may comprise a hydrogel material. Typically, hydrogel materials are polymeric materials which are capable of absorbing at least 10% by weight of water when fully hydrated. Hydrogel materials include polyvinyl alcohol (PVA), modified PVA (e.g. nelfilcon A), poly(hydroxyethyl methacrylate), poly(vinyl pyrrolidone), PVA with a poly(carboxylic acid) (e.g. carbopol), poly(ethylene glycol), polyacrylamide, polymethacrylamide, silicone-containing hydrogels, polyurethane, polyurea, and the like.

Alternatively, the ophthalmic device may be an implantable ophthalmic device. Glucose levels in tears may be much lower than blood glucose levels. With an implantable ophthalmic sensor, one can monitor glucose levels in aqueous humor or interstitial fluid, where glucose levels can be much higher than glucose levels in tears. Preferably, the device is in the form of a subconjunctive implant, intracorneal lens, stent or glaucoma shunt.

Particularly when the analyte is glucose or lactate, it is preferred that the lens outer comprises a catalyst of the invention. In this way, it may be possible to block the interference of a component other than the analyte, which interacts with the medium.

The method of the invention may be used to authenticate an article. Where the holographic element is a sensor, the sensor may be applied to an article using a transferable holographic film which is, for example, provided on a hot stamping tape. The article may be a transaction card, banknote, passport, identification card, smart card, driving licence, share certificate, bond, cheque, cheque card, tax banderole, gift voucher, postage stamp, rail or air ticket, telephone card, lottery card, event ticket, credit or debit card, business card, or an item used in consumer, brand and product protection for the purpose of distinguishing genuine products from counterfeit products and identifying stolen products. The sensors may be used to provide product and pack information for intelligent packaging applications. “Intelligent packaging” refers to a system that comprises part of, or an attachment to, a container, wrapper or enclosure, to monitor, indicate or test product information or quality or environmental conditions that will affect product quality, shelf life or safety and typical applications, such as indicators showing time-temperature, freshness, moisture, alcohol, gas, physical damage and the like.

Alternatively, the sensors can be applied to products with a decorative element or application such as any industrial or handicraft item including but not limited to items of jewellery, items of clothing (including footwear), fabric, furniture, toys, gifts, household items (including crockery and glassware), architecture (including glass, tile, paint, metals, bricks, ceramics, wood, plastics and other internal and external installations), art (including pictures, sculpture, pottery and light installations), stationery (including greetings cards, letterheads and promotional material) and sporting goods.

The invention is particularly relevant to a diagnostic device such as a test strip, chip, cartridge, swab, tube, pipette or any form of liquid sampling or testing device, and products or processes relating to human or veterinary prognostics, theranostics, diagnostics or medicines. The sensors may be used in a contact lens, sub-conjuctival implant, sub-dermal implant, test strip, chip, cartridge, swab, tube, breathalyser, catheter, any form or blood, urine or body fluid sampling or analysis device. The sensors may also be used in a product or process relating to petrochemical and chemical analysis and testing, for example in a testing device such as a test strip, chip, cartridge, swab, tube, pipette or any form of liquid sampling or analysis device.

The present invention also extends to a product suitable for use in the method of the invention comprising a holographic element where the product is capable of generating data from the holographic element and to a system which uses the data for data storage, control, transmission, reporting and/or modelling.

The following Examples illustrate the invention, the exception being Example 1, which illustrates features of the invention.

In the Examples, a holographic sensor comprising a polymeric support medium containing 12 mol % 3-acrylamidophenylboronic acid (the synthesis of which is described in WO2004/081624). The α- and β-D-glucopyranose forms of glucose were obtained from Sigma in solid form. Mutarotase was purchased from Biozyme and originated from porcine kidney. Glucose isomerase was obtained from Hampton Research and originated from Streptomyces rubiginosus. Lactate oxidase was purchased from Sigma and originated from Pediococcus sp. Detection took place in PBS, pH 7.4 at 30° C.

### EXAMPLE 1

Freshly-dissolved α-glucopyranose was detected using a holographic sensor and the rate of binding recorded. Also, a solution of α-glucopyranose was left overnight to equilibrate, and the rate of binding then determined. The experiment was repeated using β-glucopyranose. The rate of reaction was calculated by determining the time taken for the holographic sensor to reach 50% of its final equilibrium peak diffraction wavelength (i.e. the half/life) using 2 mM of the solutions.

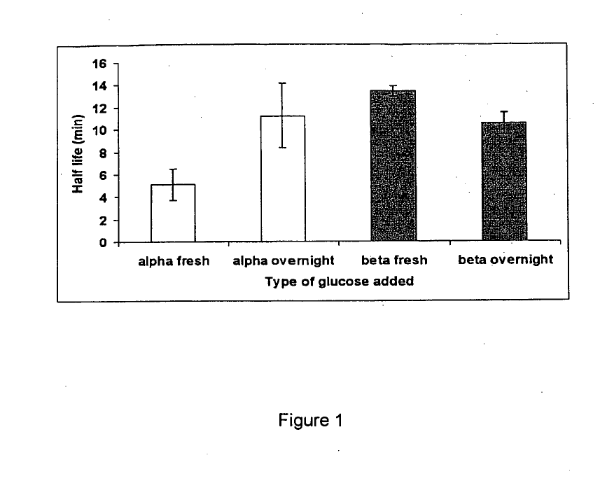

Results are shown in FIG. 1. It is evident that the freshly-dissolved α-glucopyranose form binds to the pendant phenylboronic acid group at a faster rate than freshly-dissolved β-glucopyranose. In the case of the two solutions left overnight, the rates were almost identical. These results suggest that the sensor binds the α-glucopyranose form more readily than the β-glucopyranose form. The similar rates observed for the solutions left overnight suggests an equilibrium effect, i.e. the β-form is converting into the α-form. The interconversion between the two forms is very slow and is likely to account for the slow binding kinetics observed.

### EXAMPLE 2

A 2 mM glucose solution was made up and left overnight to equilibrate. A holographic sensor was then used to detect glucose in the presence of varying amounts of mutarotase. The initial rate of response, i.e. the initial increase in peak diffraction wavelength upon addition of the glucose solution, was determined.

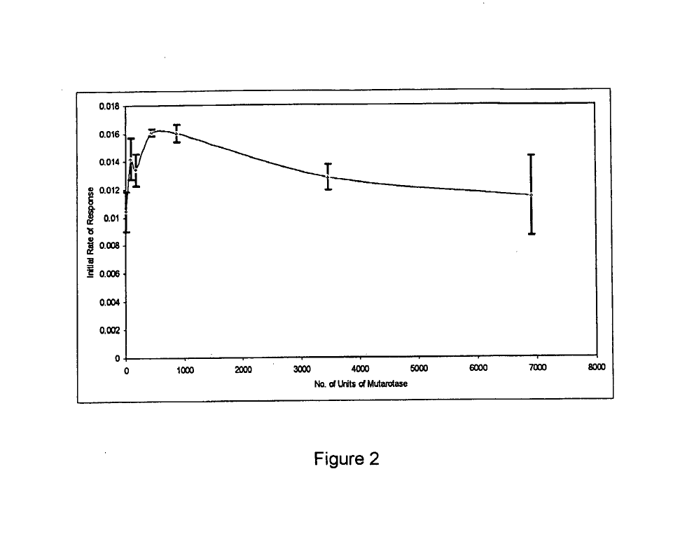

The results are shown in FIG. 2 and indicate that, at relatively lower concentrations of mutarotase, the initial rate of binding is faster than when mutarotase is absent. The optimum amount of mutarotase was found to be 0.25 mg/ml, which increased the rate of reaction by 54% relative to the control.

### EXAMPLE 3

The effect of glucose isomerase on the binding of glucose to a holographic sensor was determined. Dialysis of glucose isomerase was performed to remove the buffer that it was suspended in. The holographic sensor allowed to equilibrate with 1 mM MgSO₄, Mg²⁺ being a co-factor for glucose isomerase. A 0.5 mM glucose solution was then added to the sensor in the presence of varying amounts of glucose isomerase.

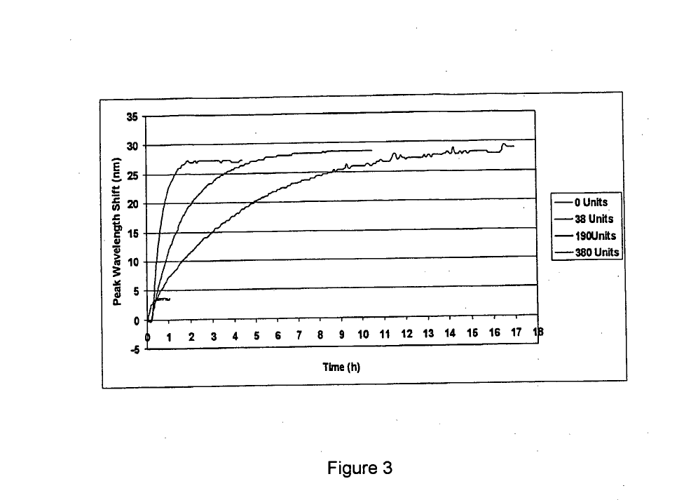

Results are shown in FIG. 3. It can be seen that the addition of glucose isomerase enhances the sensitivity of the sensor. It is also noticeable that, the greater the quantity of glucose isomerase added, the longer the system takes to equilibrate. The initial rates of reaction are also much faster than that of the control.

### EXAMPLE 4

A holographic sensor was placed in a cuvette with PBS, and 12.5 units of lactate oxidase added. Once the system had equilibrated, 2 mM lactate solution was added and the shift in peak diffraction wavelength detected over time.

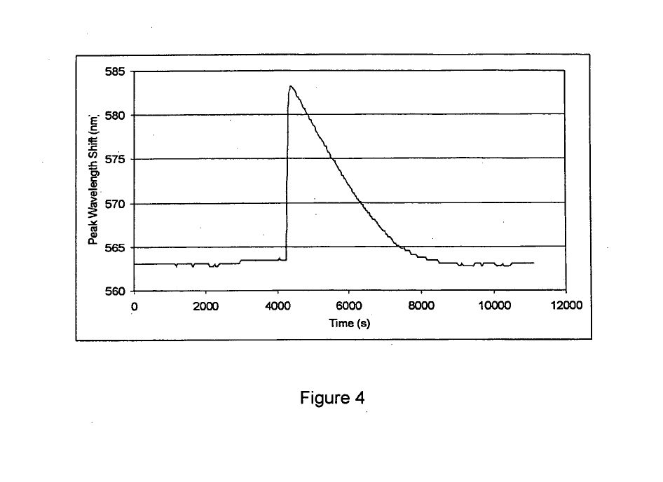

The results are shown in FIG. 4. Initially, the support medium of the sensor swelled up as it bound lactate but then contracted as lactate began to be consumed by lactate oxidase. The peak wavelength eventually returned to its initial value, indicating that all the lactate had been converted to pyruvate.

1. A method for the detection of an analyte in a fluid, which comprises contacting the fluid with a holographic element comprising a medium and a hologram disposed throughout the volume of the medium, wherein an optical characteristic of the element changes as a result of a variation of a physical property occurring throughout the volume of the medium, and wherein the variation arises as a result of interaction between the medium and the analyte; and detecting any change of the optical characteristic of the element; wherein
(a) the medium comprises a group which is capable of reacting with the analyte, wherein the analyte or the group is capable of existing in a plurality of forms, and the detecting is conducted in the presence of a first catalyst which is capable of catalysing the conversion of a relatively less reactive form of the analyte or group to a relatively more reactive form; or
(b) the fluid comprises a component, other than the analyte, which is capable of interacting with the medium, and the detecting is conducted in the presence of a second catalyst capable of catalysing the removal of said component.

2. The method according to claim 1, wherein the analyte is glucose.

3. The method according to claim 2, wherein condition (a) applies, the first catalyst is mutarotase or glucose isomerase, and the group is a phenylboronic acid group or a derivative thereof.

4. The method according to claim 2, wherein condition (b) applies, said component is lactate and the second catalyst is lactate oxidase or lactate dehydrogenase.

5. The method according to claim 4, wherein the second catalyst is lactate oxidase and detection takes place in the presence of catalase.

6. The method according to claim 1, wherein the analyte is lactate.

7. The method according to claim 6, wherein condition (b) applies, said component is glucose and the second catalyst is glucose oxidase.

8. The method according to claim 1, wherein the contacting comprises passing the fluid continuously over the element.

9. The method according to claim 1, wherein the hologram is generated by the diffraction of light.

10. The method according to claim 1, wherein the hologram is only visible under magnification.

11. The method according to claim 1, wherein the holographic image is of an object or is a 2- or 3-dimensional effect.

12. The method according to claim 1, wherein the holographic element further comprising means for producing an interference effect when illuminated with laser light.

13. The method according to claim 12, wherein the means comprises a depolarising layer.

14. The method according to claim 1, wherein the hologram is viewable under white light, UV light or infra-red radiation.

15. The method according to claim 1, wherein the hologram is viewable under specific temperature, magnetism or pressure conditions.

16. An ophthalmic device which is suitable for use in a method for the detection of an analyte in a fluid, and wherein said method comprises contacting the fluid with a holographic element comprising a medium and a hologram disposed throughout the volume of the medium, wherein an optical characteristic of the element changes as a result of a variation of a physical property occurring throughout the volume of the medium, and wherein the variation arises as a result of interaction between the medium and the analyte, and detecting any change of the optical characteristic of the element; wherein
(a) the medium comprises a group which is capable of reacting with the analyte, wherein the analyte or the group is capable of existing in a plurality of forms, and the detecting is conducted in the presence of a first catalyst which is capable of catalysing the conversion of a relatively less reactive form of the analyte or group to a relatively more reactive form; or
(b) the fluid comprises a component, other than the analyte which is capable of interacting with the medium and the detecting is conducted in the presence of a second catalyst capable of catalysing the removal of said component;
and wherein said ophthalmic device comprises a holographic element and a first or second catalyst.

17. The device according to claim 9, which is a contact lens.

18. The device according to claim 10, wherein the lens outer comprises the catalyst.

19. An article suitable for use in a method for the detection of an analyte in a fluid, wherein said method comprises contacting the fluid with a holographic element comprising a medium and a hologram disposed throughout the volume of the medium, wherein an optical characteristic of the element changes as a result of a variation of a physical property occurring throughout the volume of the medium, and wherein the variation arises as a result of interaction between the medium and the analyte; and detecting any change of the optical characteristic of the element; wherein
(a) the medium comprises a group which is capable of reacting with the analyte, wherein the analyte or the group is capable of existing in a plurality of forms, and the detecting is conducted in the presence of a first catalyst which is capable of catalysing the conversion of a relatively less reactive form of the analyte or group to a relatively more reactive form; or
(b) the fluid comprises a component, other than the analyte, which is capable of interacting with the medium, and the detecting is conducted in the presence of a second catalyst capable of catalysing the removal of said component;
and wherein said article comprises, a holographic element.

20. The article according to claim 19, which is a transaction card, banknote, passport, identification card, smart card, driving license, share certificate, bond, cheque, cheque card, tax banderole, gift voucher, postage stamp, rail or air ticket, telephone card, lottery card, event ticket, credit or debit card, business card, or an item used in consumer, brand or product protection for the purpose of distinguishing genuine products from counterfeit products or identifying stolen products.

21. The article according to claim 19, which is an item of intelligent packaging.

22. The article according to claim 19, which is an industrial or handicraft item comprising a decorative element, selected from items of jewellery, items of clothing, fabric, furniture, toys, gifts, household items, architecture, art, stationery and sporting goods.

23. The article according to claim 19, which is a product or device for use in agricultural studies, environmental studies, human or veterinary prognostics, theranostics, diagnostics, therapy or chemical analysis.

24. The article according to claim 23, which is a test strip, chip, cartridge, swab, tube, pipette, contact lens, sub-conjuctival implant, sub-dermal implant, breathalyser, catheter or a fluid sampling or analysis device.

25. A transferable holographic film for use in a method for the detection of an analyte in a fluid, wherein said method comprises contacting the fluid with a holographic element comprising a medium and a hologram disposed throughout the volume of the medium, wherein an optical characteristic of the element changes as a result of a variation of a physical property occurring throughout the volume of the medium, and wherein the variation arises as a result of interaction between the medium and the analyte; and detecting any change of the optical characteristic of the element; wherein
(a) the medium comprises a group which is capable of reacting with the analyte, wherein the analyte or the group is capable of existing in a plurality of forms, and the detecting is conducted in the presence of a first catalyst which is capable of catalysing the conversion of a relatively less reactive form of the analyte or group to a relatively more reactive form; or
(b) the fluid comprises a component, other than the analyte, which is capable of interacting with the medium, and the detecting is conducted in the presence of a second catalyst capable of catalysing the removal of said component;
and wherein said film comprises a holographic element and a first or second catalyst.

26. The film according to claim 25, which is present on a hot stamping tape.

27. A method of enhancing the security of an article, wherein said method comprises transferring onto the article the holographic element from a transferable holographic film wherein said film is suitable for use in a method for the detection of an analyte in a fluid, wherein said method for detection comprises contacting the fluid with a holographic element comprising a medium and a hologram disposed throughout the volume of the medium, wherein an optical characteristic of the element changes as a result of a variation of a physical property occurring throughout the volume of the medium, and wherein the variation arises as a result of interaction between the medium and the analyte; and detecting any change of the optical characteristic of the element; wherein
(a) the medium comprises a group which is capable of reacting with the analyte, wherein the analyte or the group is capable of existing in a plurality of forms, and the detecting is conducted in the presence of a first catalyst which is capable of catalysing the conversion of a relatively less reactive form of the analyte or group to a relatively more reactive form; or
(b) the fluid comprises a component, other than the analyte, which is capable of interacting with the medium, and the detecting is conducted in the presence of a second catalyst capable of catalysing the removal of said component;
and wherein said film comprises a holographic element and a first or second catalyst.

28. A product suitable for use in a method for the detection of an analyte in a fluid, wherein said method comprises contacting the fluid with a holographic element comprising a medium and a hologram disposed throughout the volume of the medium, wherein an optical characteristic of the element changes as a result of a variation of a physical property occurring throughout the volume of the medium, and wherein the variation arises as a result of interaction between the medium and the analyte, and detecting any change of the optical characteristic of the element; wherein
(a) the medium comprises a group which is capable of reacting with the analyte, wherein the analyte or the group is capable of existing in a plurality of forms, and the detecting is conducted in the presence of a first catalyst which is capable of catalysing the conversion of a relatively less reactive form of the analyte or group to a relatively more reactive form; or
(b) the fluid comprises a component, other than the analyte which is capable of interacting with the medium, and the detecting is conducted in the presence of a second catalyst capable of catalysing the removal of said component;
and wherein said product comprises a holographic element that is capable of generating data from said element.

29. A system which uses data generated by a product for data storage, control, transmission, reporting and/or modeling wherein said product is suitable for use in the detection of an analyte in a fluid, which comprises contacting the fluid with a holographic element comprising a medium and a hologram disposed throughout the volume of the medium, wherein an optical characteristic of the element changes as a result of a variation of a physical property occurring throughout the volume of the medium, and wherein the variation arises as a result of interaction between the medium and the analyte; and detecting any change of the optical characteristic of the element; wherein
(a) the medium comprises a group which is capable of reacting with the analyte, wherein the analyte or the group is capable of existing in a plurality of forms, and the detecting is conducted in the presence of a first catalyst which is capable of catalysing the conversion of a relatively less reactive form of the analyte or group to a relatively more reactive form; or
(b) the fluid comprises a component, other than the analyte, which is capable of interacting with the medium, and the detecting is conducted in the presence of a second catalyst capable of catalysing the removal of said component;
and wherein said product comprises a holographic element that is capable of generating data from said element.
